(19) United States
(12) Patent Application Publication
(10) Pub. No.: US 2025/0154018 A1
(43) Pub. Date: May 15, 2025

(54) COATED CERIUM SUBOXIDE PARTICLES AND PREPARATION THEREOF BY FLAME SPRAY PYROLYSIS

(71) Applicant: L'Oreal, Paris (FR)

(72) Inventors: Valérie Jeanne-Rose, AULNAY-SOUS-BOIS (FR); Henri Samain, CHEVILLY-LARUE (FR); Maria Louloudi, IOANNINA (GR); Yiannis Deligiannakis, IOANNINA (GR)

(73) Assignee: L'Oreal, Paris (FR)

(21) Appl. No.: 18/722,340

(22) PCT Filed: Dec. 20, 2022
(86) PCT No.: PCT/EP2022/087057

§ 371 Date: Jun. 20, 2024

(30) Foreign Application Priority Data
Dec. 21, 2021 (FR) FR2114169

(51) Int. Cl. C01F 17/235; A61K 8/19

(52) U.S. Cl. CPC C01F 17/235; A61K 8/19; A61K 2800/621; A61K 2800/651; C01P 2004/61; C01P 2004/62; C01P 2004/64; C01P 2004/84

(57) ABSTRACT

The present invention relates to coated cerium suboxide particles, to a process for preparing coated cerium oxide or suboxide particles by means of flame spray pyrolysis technology, to the cerium oxide particles resulting from such a process, to compositions comprising such particles and also to the uses thereof.

The present invention relates to coated cerium suboxide particles, to a process for preparing coated cerium oxide or suboxide particles by means of flame spray pyrolysis technology, to the cerium oxide particles resulting from such a process, to compositions comprising such particles and also to the uses thereof.

Metal oxides are used in many applications (cosmetics, paints, varnishes, electronics, rubber, etc.), notably for their optical properties. In particular, use is made of their light absorption and/or light scattering properties in order to protect surfaces from UV radiation and/or in order to convert ambient light into electricity.

However, metal oxides have the drawback of being particularly unstable over time. By way of example, zinc oxide may degrade to zinc hydroxide, or even to Zn²⁺ ion, in the presence of water originating from the composition comprising it or from atmospheric moisture. Such a degradation leads to partial or even total dissolution of the zinc oxide in water and has the effect of greatly reducing, or even eliminating, the desired properties of the zinc oxide.

This instability is particularly problematic when the metal oxides are used in photoprotective cosmetic compositions. Indeed, the ultraviolet radiation protection decreases as the metal oxides degrade.

It has been envisaged to coat the metal oxides with silica, notably using sol-gel processes, or else to graft fluoro compounds onto the metal oxides. However, these solutions are not entirely satisfactory. The metal oxides coated with silica via a sol-gel process generally have poorer optical properties than those of an uncoated particle. As for the grafting technique, the use of fluoro compounds can be harmful to the environment and hazardous to the user.

It is also known practice to use a Flame Spray Pyrolysis (FSP) method to prepare metal oxide particles.

Flame spray pyrolysis or FSP is a method that is nowadays well known, which was essentially developed for the synthesis of ultrafine powders of single or mixed oxides of various metals (e.g. SiO₂, Al₂O₃, B₂O₃, ZrO₂, GeO₂, WO₃, Nb₂O₅, SnO₂, MgO, ZnO), having controlled morphologies, and/or their deposition on various substrates, this being performed starting from a wide variety of metal precursors, generally in the form of organic or inorganic, preferably inflammable, sprayable liquids; the liquids sprayed into the flame, on being consumed, notably emit metal oxide nanoparticles which are projected by the flame itself onto these various substrates. The principle of this method was recalled, for example, in the recent (2011) Johnson Matthey publication entitled “Flame Spray Pyrolysis: a Unique Facility for the Production of Nanopowders”, Platinum Metals Rev., 2011, 55 (2), 149-151. Numerous variants of FSP processes and reactors have also been described, by way of example, in the following patents or patent applications: U.S. Pat. Nos. 5,958,361, 2,268,337, WO 01/36332 or U.S. Pat. No. 6,887,566, WO 2004/005184 or U.S. Pat. No. 7,211,236, WO 2004/056927, WO 2005/103900, WO 2007/028267 or U.S. Pat. No. 8,182,573, WO 2008/049954 or U.S. Pat. No. 8,231,369, WO 2008/019905, US 2009/0123357, US 2009/0126604, US 2010/0055340, WO 2011/020204.

However, this method, applied to the preparation of metal oxide, remains to be perfected, notably so as to improve the stability of the metal oxide particles over time, and more particularly, their water resistance. More particularly, these preparation processes do not make it possible to obtain, easily and in large number, oxides of metals with an intermediate oxidation number or metal suboxides. Furthermore, the oxides of metals with an intermediate oxidation number and metal suboxides prepared according to these known processes are not stable over time and oxidize to give their maximum oxidation number very rapidly on contact with ambient air.

There is thus a real need to develop metal oxide particles which have good stability over time, and most particularly good water resistance, while at the same time maintaining good optical properties in terms of absorption and/or scattering of light, more particularly of ultraviolet radiation; and also to develop a process that is capable of preparing such particles.

In addition, it is also of interest to develop a process for preparing such particles, and in particular for preparing particles of metal oxides of intermediate oxidation number and metal suboxides which have good stability over time and good optical properties in terms of absorption and/or scattering of light, more particularly ultraviolet radiation.

These aims are achieved with the present invention, one subject of which is notably cerium suboxide particles, in particular of the Ce-M′ suboxide type with a core/shell structure, comprising:
(i) a core 1 constituted of at least one cerium suboxide of formula (I′):

CeO₂₋ₓ (I′)
in which:
   x represents a non-integer number strictly between 0 and 2; and
(ii) an upper coating layer 2, covering the surface of said core 1, constituted of at least one compound of formula (II):

M′ₚO_{q} (II)
in which:
   M′ is an element chosen from selenium and the elements in columns 4, 13 and 14 of the Periodic Table of the Elements;
   p represents an integer greater than or equal to 1,
   q represents an integer greater than or equal to 0.

It has been found that the coated cerium oxide particles according to the invention degrade very little over time in the presence of water, even when they are formulated in a composition, notably an aqueous composition.

More particularly, it has been observed that the cerium suboxide particles according to the invention are particularly stable over time (i.e. the particles remain in their suboxide state).

It has also been found that the cerium oxide particles according to the invention have good optical properties in terms of absorption and/or scattering of light. More particularly, they have high UV absorption and low visible scattering or high visible scattering, then permitting uses such as sun protection and/or modification of the visual appearance, while benefiting from resistance in the presence of water.

In addition, the compositions comprising coated cerium oxide particles according to the invention have shown good screening power, notably with respect to long and short UV-A radiation.

Moreover, the compositions comprising the coated cerium oxide particles of the invention have particularly high transparency, which may prove to be advantageous when the composition is applied and then left to dry on the coating, and in particular on the skin.

In addition, since the coated cerium oxide particles according to the invention do not require a hydrophobic coating, it is possible to use them in a wide spectrum of formulations (for example in fully aqueous formulations and/or surfactant-free formulations). When the formulations thus obtained end up in water (washbasin drainage, lake or sea), the risk of inappropriate deposition (on the edges of the washbasin, on the walls of the pipes or on rocks) is furthermore reduced.

The invention also relates to a process for preparing cerium oxide or suboxide particles coated with an oxide of an element M′, in particular of the Ce-M′ (sub)oxide type with a core/shell structure, comprising:
(i) a core 1 constituted of at least one cerium oxide of formula (I):

CeO₂₋ₓ (I)
in which:
   x is equal to 0 or represents a non-integer number strictly between 0 and 2; and
(ii) an upper coating layer 2, covering the surface of said core 1, constituted of at least one compound of formula (II):

M′ₚO_{q} (II)
in which:
   M′ is an element chosen from selenium and the elements in columns 4, 13 and 14 of the Periodic Table of the Elements;
   p represents an integer greater than or equal to 1;
   q represents an integer greater than or equal to 0;
and comprising at least the following steps:
a. preparing a composition (A) by adding one or more cerium precursors in a combustible solvent or in a mixture of combustible solvents; and then
b. in a flame spray pyrolysis device 10, forming a flame by injecting composition (A) and an oxygen-containing gas (G) until cerium oxide aggregates of formula (I) are obtained; and then
c. injecting a composition (B) comprising one or more precursors of element M′ until an upper coating layer 2 constituted of element M′ or of element M′ oxide of formula (II) is obtained on the surface of said cerium oxide aggregates; and
said flame spray pyrolysis device 10 being isolated from the outside air, so that the amount of oxygen present in said device is controlled.

It has been found that the process according to the invention makes it possible to obtain cerium oxide and suboxide particles coated with a layer of inorganic material based on element M′, which are particularly stable over time and have good water resistance.

Moreover, unlike conventional coating processes, the process according to the invention has the advantage, despite the presence of an upper coating layer, of maintaining good intrinsic performance qualities of the core. Indeed, due to the particular nature of the upper coating layer, it is possible for a given particle weight to reduce the proportion of cerium oxide or suboxide, without, however, reducing and/or negatively affecting the properties of said cerium oxide or suboxide.

Thus, the process of the invention makes it possible to prepare stable cerium oxide and suboxide particles, while at the same time avoiding the drawbacks due to the increase in the amount of particles that would conventionally be necessary to maintain the good optical properties of the cerium oxide or suboxide.

The invention also relates to a composition, preferably a cosmetic composition, comprising one or more cerium suboxide particles according to the invention.

### BRIEF DESCRIPTION OF THE FIGURES

The present invention will be understood more clearly on studying the detailed description of embodiments, given by way of examples which are not at all limiting and illustrated by the appended drawings, not necessarily to scale, in which:

FIG. 1 is a cross-sectional view of a cerium suboxide particle of formula (I′) coated with a compound of formula (II) according to one embodiment of the invention;

FIG. 2 is a schematic view of a flame spray pyrolysis device for preparing the particles according to the invention; and

FIG. 3 is a schematic view of a flame spray pyrolysis device for preparing the particles according to another embodiment of the invention.

Other characteristics, aspects and advantages of the invention will emerge even more clearly on reading the description and the example which follows.

In the present description, and unless otherwise indicated:
the expression “at least one” is equivalent to the expression “one or more” and can be replaced therewith;
the expression “between” is equivalent to the expression “ranging from” and can be replaced therewith, and implies that the limits are included;
the expression “strictly between” is equivalent to the expression “strictly ranging from” and can be replaced therewith, and implies that the limits are not included;
the expression “keratin materials” in particular denotes the skin and human keratin fibres, such as the hair;
core 1 is also called the “heart” or the “core”;
the upper coating layer 2 is also referred to as the “outer layer”, “casing”, “coating” or “shell”;
for the purposes of the present invention, the term “elements of column 3 of the Periodic Table of the Elements” means scandium and yttrium. In other words, the elements of the lanthanide family and of the actinide family do not belong to the elements of column 3 of the Periodic Table of the Elements within the meaning of the invention;
the term “alkyl” means an “alkyl radical”, that is to say a linear or branched C₁ to C₁₀, particularly C₁ to C₈, more particularly C₁ to C₆ and preferentially C₁ to C₄ hydrocarbon-based radical, such as methyl, ethyl, propyl, isopropyl, n-butyl, isobutyl or tert-butyl;
the term “aryl” radical means a monocyclic or fused or non-fused polycyclic carbon-based group comprising from 6 to 22 carbon atoms, at least one ring of which is aromatic; preferentially, the aryl radical is a phenyl, biphenyl, naphthyl, indenyl, anthracenyl or tetrahydronaphthyl, preferably a phenyl;
the term “arylate” radical means an aryl group which comprises one or more carboxylate —C(O) O⁻ groups, such as naphthalate or naphthenate;
the term “complexed metal” means that the metal atom forms a “metal complex” or “coordination compound” in which the metal ion, corresponding to the central atom, i.e. M, is chemically bonded to one or more electron donors (ligands);
the term “ligand” means a coordinating organic chemical group or compound, i.e. which comprises at least one carbon atom and which is capable of coordination with the metal M, and which, once coordinated or complexed, results in metal compounds corresponding to principles of a coordination sphere with a predetermined number of electrons (internal complexes or chelates)—see Ullmann's Encyclopedia of Industrial Chemistry, “Metal-Complex Dyes”, 2005, pages 1-42. More particularly, the ligand(s) are organic groups which include at least one group which is electron-donating via an inductive and/or mesomeric effect, more particularly bearing at least one amino, phosphino, hydroxyl or thiol electron-donating group, or the ligand is a persistent carbene, particularly of “Arduengo” type (imidazol-2-ylidenes), or includes at least one carbonyl group. Mention may be made more particularly, as ligand, of: i) those which contain at least one phosphorus-P<atom, i.e. phosphine, such as triphenylphosphines; ii) bidendate ligands of formula R—C(X)—CR′R″—C(X)—R′″ with R and R′″, which may be identical or different, representing a linear or branched (C₁-C₆)alkyl group and R′ and R″, which may be identical or different, representing a hydrogen atom or a linear or branched (C₁-C₆)alkyl group, preferentially R′ and R″ representing a hydrogen atom, X representing an oxygen or sulfur atom or a group N(R) with R representing a hydrogen atom or a linear or branched (C₁-C₆)alkyl group, such as acetylacetone or β-diketones; iii) (poly)hydroxycarboxylic acid ligands of formula [HO—C(O)]ₙ-A-C(O)—OH and their deprotonated forms with A representing a monovalent group when n has the value zero or a polyvalent group when n is greater than or equal to 1, which is saturated or unsaturated, cyclic or non-cyclic and aromatic or non-aromatic based on a hydrocarbon comprising from 1 to 20 carbon atoms which is optionally interrupted with one or more heteroatoms and/or is optionally substituted, notably with one or more hydroxyl groups: preferably, A represents a monovalent (C₁-C₆)alkyl group or a polyvalent (C₁-C₆)alkylene group optionally substituted with one or more hydroxyl groups; and n representing an integer between 0 and 10 inclusive; preferably, n is between 0 and 5, such as between 0, 1 or 2; such as lactic, glycolic, tartaric, citric and maleic acids, and arylates, such as naphthalates; and iv) C₂ to Co polyol ligands comprising from 2 to 5 hydroxyl groups, notably ethylene glycol or glycerol; even more particularly, the ligand(s) bear a carboxyl, carboxylate or amino group; particularly, the ligand is chosen from acetate, (C₁-C₆)alkoxylate, (di)(C₁-C₆)alkylamino and arylate, such as naphthalate or naphthenate, groups;
the term “combustible” means a liquid compound or a gas which, with dioxygen and energy, is consumed in a heat-generating chemical reaction: combustion. In particular, the liquid combustibles are chosen from protic solvents, in particular alcohols, such as methanol, ethanol, ispropanol or n-butanol; aprotic solvents, in particular chosen from esters, such as methyl esters and those resulting from acetate, such as 2-ethylhexyl acetate, acids, such as 2-ethylhexanoic acid (EHA), acyclic ethers, such as ethyl ether, methyl tert-butyl ether (MTBE), methyl tert-amyl ether (MTAE), methyl tert-hexyl ether (MTHE), ethyl tert-butyl ether (ETBE), ethyl tert-amyl ether (ETAE) or diisopropyl ether (DIPE), cyclic ethers, such as tetrahydrofuran (THF), aromatic hydrocarbons or arenes, such as xylene, non-aromatic hydrocarbons; and mixtures thereof. The combustibles may optionally be chosen from liquefied hydrocarbons, such as acetylene, methane, propane or butane; and mixtures thereof.

### Coated Cerium Suboxide Particles

The cerium suboxide particles, in particular of the Ce-M′ suboxide type with a core/shell structure, comprise:
(i) a core 1 constituted of at least one cerium suboxide of formula (I′):

CeO₂₋ₓ (I′)
in which:
   x represents a non-integer number strictly between 0 and 2; and
(ii) an upper coating layer 2, covering the surface of said core 1, constituted of at least one compound of formula (II):

M′ₚO_{q} (II)
in which:
   M′ is an element chosen from selenium and the elements in columns 4, 13 and 14 of the Periodic Table of the Elements;
   p represents an integer greater than or equal to 1;
   q represents an integer greater than or equal to 0.

According to the invention, the non-integer number x in formula (I′) is strictly comprised between 0 and 2. Thus, x cannot be 0 (zero) or 2 in formula (I′). In other words, the cerium oxide CeO₂ (x=0) and the pure element cerium (x=2) do not fall within the scope of cerium suboxides of formula (I′).

Preferably, the core 1 is in a crystalline state.

The crystalline state of the core 1 and its composition can be determined, for example, by a conventional X-ray diffraction method.

Advantageously, the core 1 of the particle according to the invention consists of one or more aggregates of crystalline primary cerium suboxide particles. In other words, the core 1 consists of several cerium suboxide microcrystals.

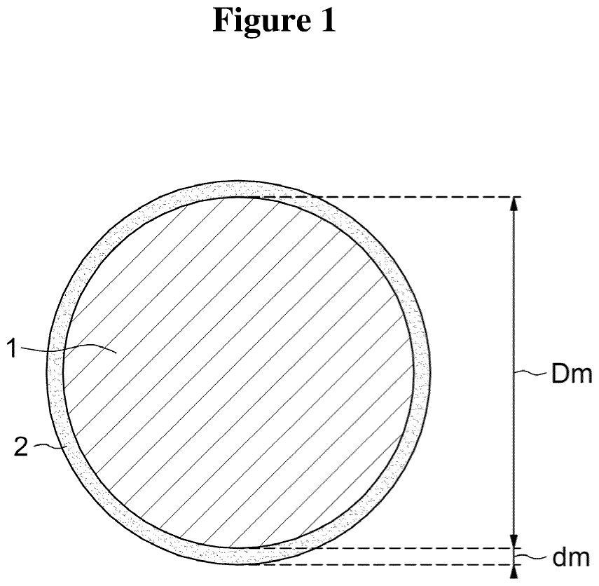

The coated cerium suboxide particle according to FIG. 1 comprises a core 1 of average diameter Dₘ, constituted of a cerium suboxide of formula (I′).

The coated cerium suboxide particle according to FIG. 1 also comprises an upper coating layer 2, constituted of a compound of formula (II), and completely covering the surface of the core 1 and of average thickness dₘ.

The number-average diameter Dₘ of the core 1 can be determined, for example, by transmission electron microscopy (abbreviated to TEM). Preferably, the number-average diameter Dₘ of the core 1 of the particles according to the invention is within the range from 3 to 5000 nm, more preferentially from 10 to 3000 nm, and even more preferentially between 30 and 1000 nm.

The coated cerium suboxide particle according to the invention comprises an upper coating layer 2, covering the surface of the core 1, constituted of a compound of formula (II).

Advantageously, the upper coating layer 2 covers at least 90% of the surface of the core 1. More preferentially, the upper coating layer 2 covers the entire surface of the core 1.

The degree of coverage of the core with the upper coating layer can be determined, for example, by means of a visual analysis of TEM-BF or STEM-HAADF type, coupled to STEM-EDX analysis.

Each of the analyses is performed on a statistical number of particles, in particular on at least 20 particles. The particles are deposited on a metal grid made of a metal other than any metal forming part of the particles, whether in the core or in the upper coating layer. For example, the grid is made of copper.

Visual analysis of the TEM-BF and STEM-HAADF images makes it possible, based on the contrast, to deduce whether or not the coating completely surrounds the core of the particle. It is possible, by analysing each of the 20 (or more) images, to deduce therefrom a degree of coverage of the core and then, by taking the average, to determine an average degree of coverage.

The STEM-EDX analysis makes it possible to check that the coating indeed contains predominantly or exclusively the element M′. For this, it is necessary to make pointings (on at least 20 particles), on the edges of the particles. These pointings then reveal the element M′.

STEM-EDX analysis can also verify that the core contains cerium. For this, it is necessary to make pointings (on at least 20 particles), at the centres of the particles. These pointings then reveal the cerium and the element M′.

According to the invention, the element M′ is chosen from selenium and the elements in columns 4, 13 and 14 of the Periodic Table of the Elements.

According to the invention, the element M′ is thus different from cerium.

Preferably, the element M′ is chosen from selenium, titanium, aluminium and the elements in column 14 of the Periodic Table of the Elements.

More preferentially, the element M′ is chosen from selenium, titanium, aluminium, carbon and silicon.

Most particularly preferably, the element M′ is chosen from carbon and silicon.

According to a preferred embodiment, the element M′ is silicon.

According to another embodiment of the invention, the element M′ is carbon.

Preferably, the integer p ranges from 1 to 4. More preferentially, the integer p is equal to 1 or 2, and better still p is equal to 1.

Preferably, the integer q ranges from 0 to 4. More preferentially, the integer q is strictly greater than 0. Even more preferentially, the integer q ranges from 1 to 4.

Preferably, the compound(s) of formula (II) are chosen from carbon, SiO₂, SnO₂ and Al₂O₃.

More preferentially, the compound(s) of formula (II) are chosen from carbon and SiO₂.

The cerium oxide of the formula CeO₂₋ₓ in the core 1 of the particle according to the invention has a non-stoichiometric intermediate oxidation number.

For the purposes of the invention, the term “intermediate oxidation number” means an oxidation number between 0 (not included) and the maximum oxidation number of the metal element (not included).

More generally, if the oxidation number is an integer, it is referred to as a stoichiometric intermediate oxidation number. For example, if the metal element with a stoichiometric intermediate oxidation number is iron, then the iron oxide(s) can be FeO and Fe₃O₄. As another example, if the metal element with a stoichiometric intermediate oxidation number is copper, then the copper oxide can be Cu₂O.

If the oxidation number is not an integer, the expression “non-stoichiometric intermediate oxidation number” is then used. When the metal element has a non-stoichiometric intermediate oxidation number, it is then referred to as a metal element suboxide. For example, if the metal element with a non-stoichiometric intermediate oxidation number is iron, then the iron suboxide(s) may be compounds of the formula FeO₁₋ₓ, compounds of the formula Fe₃O₄₋ₓ and compounds of the formula Fe₂O₃₋ₓ. As another example, if the metal element with a non-stoichiometric intermediate oxidation number is copper, then the copper suboxides may be compounds of the formula CuO₁₋ₓ and compounds of the formula Cu₂O₁₋ₓ.

According to a preferred embodiment of the invention, the particles comprise:
a core 1 constituted of a cerium suboxide CeO₂₋ₓ of formula (I′) where x represents a non-integer number strictly between 0 and 2; and
an upper coating layer 2, covering the surface of said core 1, constituted of a compound of formula (II) chosen from carbon, SiO₂, SnO₂ and Al₂O₃, preferentially SiO₂.

According to another particularly preferred embodiment of the invention, the particles comprise:
a core 1 constituted of a cerium suboxide CeO₂₋ₓ of formula (I′) where x represents a non-integer number strictly between 0 and 2; and
an upper coating layer 2, covering the surface of said core 1, constituted of a compound of formula (II) chosen from carbon and SiO₂, preferentially SiO₂.

The number-average thickness dₘ of the upper coating layer can also be determined by transmission electron microscopy.

Preferably, the number-average thickness dₘ of the upper coating layer is within the range from 1 to 20 nm, more preferentially from 1 to 10 nm and even more preferentially from 2 to 6 nm.

Advantageously, the upper coating layer 2 is amorphous.

Advantageously, the upper coating layer 2 is transparent.

Advantageously, the particle according to the invention comprises cerium and the element M′ in a particular molar atomic ratio (Ce/M′).

This ratio corresponds to the amount in moles of cerium present in the particle according to the invention, on the one hand, to the amount in moles of element M′ present in the particle according to the invention, on the other hand.

This ratio can be determined by spectrometry according to one of the following two methods. According to a first method, powder is spread out and an X-ray fluorimetry study is performed with an X-ray spectrometer to deduce therefrom the metal ratio. According to another method, the particles of the invention are dissolved beforehand in an acid. An elemental analysis is then performed on the obtained material by ICP-MS (Inductively Coupled Plasma Mass Spectrometry) to deduce therefrom the metal ratio.

Preferably, the molar atomic ratio (Ce/M′) of the particle according to the invention is strictly greater than 0.2; more preferentially greater than or equal to 1; even more preferentially in the range from 1 to 100; better still in the range from 1 to 10, and even better still in the range from 1.5 to 10.

The number-average diameter of the particle according to the invention can also be determined by transmission electron microscopy. Preferably, the number-average diameter of the particles according to the invention is in the range from 4 to 5000 nm; more preferentially from 10 to 3000 nm; and even more preferentially 30 to 1000 nm.

Preferably, the BET specific surface area of the particle according to the invention is between 1 m²/g and 200 m²/g, more preferentially between 30 and 100 m²/g.

According to a particular embodiment of the invention, the coated particle according to the invention may optionally also comprise an additional coating layer covering the upper coating layer 2 and comprising at least one hydrophobic organic compound.

The hydrophobic organic compound(s) included in the additional coating layer are more preferentially chosen from silicones, in particular silicones comprising at least one fatty chain; carbon-based derivatives comprising at least 6 carbon atoms, in particular fatty acid esters; and mixtures thereof.

The additional coating layer can be produced via a liquid route or via a solid route. Via a liquid route, the hydroxyl functions are reacted with reactive functions of the compound which will form the coating (typically silanol functions of a silicone or the acid functions of a carbon-based fatty substance). Via a solid route, the particles are placed in contact with a liquid or pasty compound including the hydrophobic substance.

Preferably, the coated particles according to the invention are obtained by means of the preparation process of the invention as described below.

### The Process for Preparing the Coated Particles

Another subject of the invention relates to a process for preparing cerium oxide or suboxide particles of the following formula (I) coated with an oxide of element M′, in particular of the Ce-M′ (sub)oxide type with a core/shell structure, comprising at least a step a. of preparing a composition (A), then a step b. of forming the flame, and a step c. of injecting a composition (B).

The cerium (sub)oxide particles coated with an oxide of element M′ which can be prepared by means of the preparation process according to the invention comprise:

(i) a core 1 constituted of at least one cerium oxide of formula (I):

CeO₂₋ₓ (I)
in which:
   x is equal to 0 or represents a non-integer number strictly between 0 and 2; and
(ii) an upper coating layer 2, covering the surface of said core 1, constituted of at least one compound of formula (II):

M′ₚO_{q} (II)
in which:
   M′ is an element chosen from selenium and the elements in columns 4, 13 and 14 of the Periodic Table of the Elements;
   p represents an integer greater than or equal to 1,
   q represents an integer greater than or equal to 0.

The particles of cerium (sub)oxides of formula (I) above include:
the cerium suboxide particles according to the invention comprising a core 1 constituted of a cerium suboxide CeO₂₋ₓ of formula (I′) as described previously, where x represents a non-integer number strictly between 0 and 2; and
the cerium oxide particles comprising a core 1 constituted of cerium oxide CeO₂ (x is equal to 0).

Step a. of the process according to the invention consists in preparing a composition (A), by adding one or more cerium precursors in a combustible solvent or in a mixture of combustible solvents.

The cerium precursors and combustible solvents that may be used according to the invention may be chosen from the cerium precursors and combustible solvents conventionally used in flame spray pyrolysis.

Preferably, the cerium precursors included in composition (A) are chosen from cerium (III) salts, cerium (IV) salts, and mixtures thereof.

The cerium (III) salts and cerium (IV) salts used may be in anhydrous or hydrated form.

More preferentially, the cerium precursors included in composition (A) are chosen from cerium (III) ethylhexanoate, cerium (III) acetate, cerium (III) chloride, cerium (III) nitrate, cerium (IV) sulfate, cerium (IV) naphthenate, and mixtures thereof.

More preferentially, the cerium precursor(s) included in composition (A) are chosen from cerium (III) ethylhexanoate, cerium (III) chloride, cerium (III) nitrate, and mixtures thereof.

Preferably, the combustible solvent(s) are chosen from protic combustible solvents, aprotic combustible solvents, and mixtures thereof; more preferentially from alcohols, esters, acids, acyclic ethers, cyclic ethers, aromatic hydrocarbon or arenes, non-aromatic hydrocarbons, such as liquefied hydrocarbons, for example acetylene, methane, propane or butane, and mixtures thereof; and better still from 2-ethylhexyl acetate, 2-ethylhexanoic acid (EHA), ethyl ether, methyl tert-butyl ether (MTBE), methyl tert-amyl ether (MTAE), methyl tert-hexyl ether (MTHE), ethyl tert-butyl ether (ETBE), ethyl tert-amyl ether (ETAE), diisopropyl ether (DIPE), tetrahydrofuran (THF), xylene and mixtures thereof.

In particular, the combustible solvent(s) may be chosen from aprotic combustible solvents comprising at least three carbon atoms and mixtures thereof; and better still from xylene, toluene, tetrahydrofuran, 2-ethylhexyl acetate, 2-ethylhexanoic acid (EHA) and mixtures thereof.

According to a particular embodiment of the invention, composition (A) comprises a mixture of combustible solvents, preferably comprising at least two of the following combustible solvents: 2-ethylhexanoic acid (EHA), toluene, absolute ethanol and diethylene glycol monobutyl ether.

Better still, composition (A) comprises a mixture of combustible solvents consisting of 2-ethylhexanoic acid (EHA), toluene, absolute ethanol and diethylene glycol monobutyl ether.

Even better still, composition (A) comprises a mixture of combustible solvents consisting of at least 5% by volume of 2-ethylhexanoic acid (EHA), of at least 5% by volume of toluene, of at least 5% by volume of absolute ethanol and of at least 5% by volume of diethylene glycol monobutyl ether, relative to the total volume of the mixture of combustible solvents.

Advantageously, the cerium precursor content in composition (A) is between 1% and 60% by weight, preferably between 15% and 30% by weight, relative to the total weight of composition (A).

The preparation process according to the invention also comprises a step b. of injecting composition (A) and an oxygen-containing gas (G) into a flame spray pyrolysis (FSP) device 10 to form a flame.

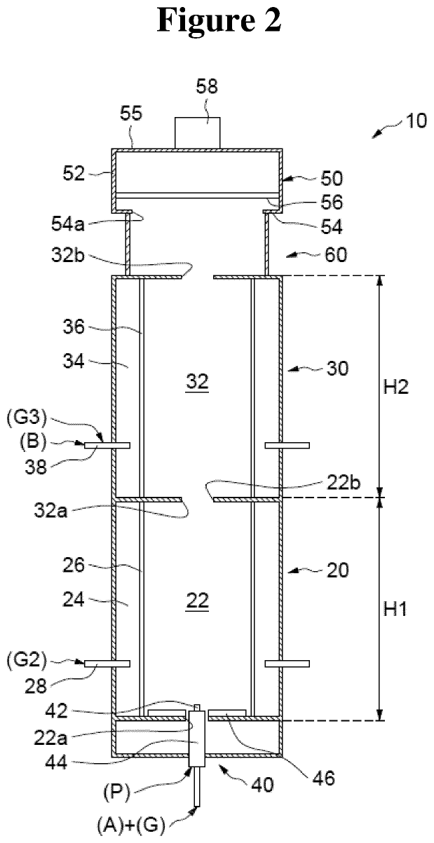

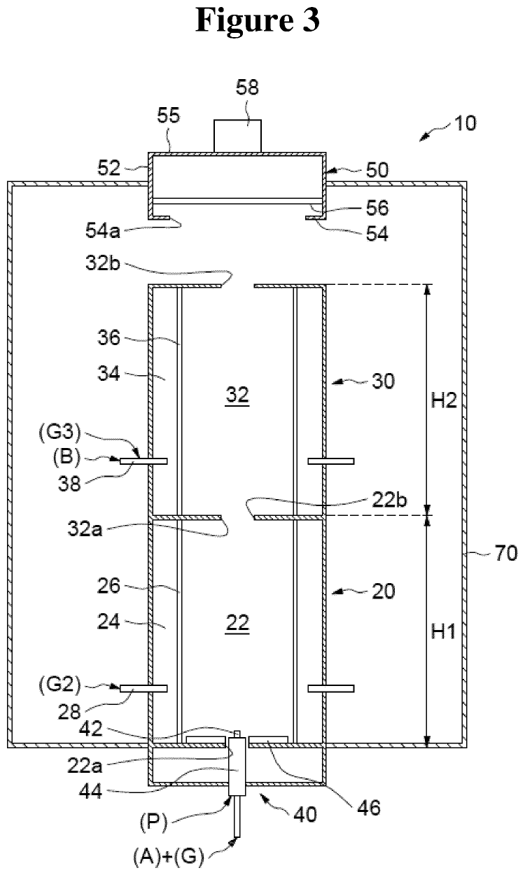

The flame spray pyrolysis device 10 will be described more specifically below with reference to FIGS. 2 and 3.

During this step b., composition (A) and the oxygen-containing gas (G) are advantageously injected into the flame spray pyrolysis device 10.

Preferably, the flame formed during step b. is at a temperature of greater than or equal to 2000° C., at at least one point in the flame.

Step b. may optionally also comprise an additional injection of a “premix” mixture (P) comprising oxygen and one or more combustible gases such as methane. This “premix” mixture (also referred to as the “supporting flame oxygen”) enables the production of a support flame intended to ignite and maintain the flame resulting from composition (A) and the oxygen-containing gas (G) (i.e. “dispersion oxygen”). The mixture of composition (A) with the gas (G), on the one hand, and the premix (P), on the other hand, are injected separately, that is to say that the mixture of composition (A) with the oxygen-containing gas (G) is injected by means of one tube and that the premix (P) is injected by means of another tube.

Preferably, during step b., composition (A), the oxygen-containing gas (G) and optionally the “premix” mixture (P), when it is present, are injected into a reaction tube (also referred to as the “enclosing tube”). Preferably, this reaction tube is made of metal or quartz. Advantageously, the reaction tube has a height of greater than or equal to 30 cm, preferably of greater than or equal to 40 cm and more preferentially of greater than or equal to 50 cm. Preferentially, the length of said reaction tube is between 30 cm and 300 cm, particularly between 40 cm and 200 cm and more particularly between 45 cm and 100 cm, such as 50 cm.

The weight ratio of the mass of solvent(s) present in composition (A), on the one hand, to the mass of oxygen-containing gas (G), on the other hand, is defined as follows:

First, the amount of oxygen-containing gas (also called the “oxidizing compound”) is calculated so that the combination of composition (A), i.e. the combustible solvent(s) and cerium precursor(s), on the one hand, and the oxygen-containing gas, on the other hand, can react together in a combustion reaction in a stoichiometric ratio (thus without any excess or deficiency of oxidizing compound).

Starting from this calculated amount of oxygen-containing gas (also referred to as the “calculated oxidizer”), a new calculation is performed to deduce therefrom the amount of oxygen-containing gas to be injected (also referred to as the “oxidizer to be injected”), according to the formula:

Oxidizer to be injected=Calculated oxidizer/φ
with φ, a correction factor, preferably between 1 and 2.2, more preferentially between 1.05 and 2, even more preferentially between 1.1 and 1.8, better still between 1.2 and 1.4.

This method is notably defined by Turns, S. R. in An Introduction to Combustion: Concepts and Applications, 3rd ed.; McGraw-Hill: New York, 2012.

Preferably, the molar amount of oxygen-containing gas (G) to be injected during step b. is strictly less than the molar amount of oxygen-containing gas required to cause composition (A) to react with the oxygen in a stoichiometric ratio.

The flame spray pyrolysis device 10 that may be used in the preparation process according to the invention may comprise one or more chambers. Preferably, the flame spray pyrolysis device 10 that may be used in the preparation process according to the invention comprises several chambers, more preferentially two chambers.

Preferably, the flame spray pyrolysis device 10 is pressurized with an inert gas (G2) chosen, for example, from nitrogen, methane, argon, hydrogen, hydrogen sulfide and ammonia; more preferentially from nitrogen, methane, hydrogen and argon; even more preferentially from nitrogen and argon, and better still nitrogen.

According to a preferred embodiment of the invention, when the flame spray pyrolysis device 10 comprises only one chamber, the chamber of said flame spray pyrolysis device 10 is pressurized with an inert gas (G2) chosen, for example, from nitrogen, methane, argon, hydrogen, hydrogen sulfide and ammonia; preferably from nitrogen, methane, hydrogen and argon; more preferentially from nitrogen and argon, and better still nitrogen.

According to another preferred embodiment of the invention, when the flame spray pyrolysis device 10 comprises several chambers, the first chamber 20 of said flame spray pyrolysis device 10 is pressurized with an inert gas (G2) chosen, for example, from nitrogen, methane, argon, hydrogen, hydrogen sulfide and ammonia; preferably from nitrogen, methane, hydrogen and argon; more preferentially from nitrogen and argon, and better still nitrogen.

Preferably, the flow rate of inert gas (G2) injected into the flame spray pyrolysis device 10 ranges from 5 L/min to 70 L/min; more preferentially from 10 L/min to 50 L/min.

More preferentially, the flow rate of nitrogen (G2) injected into the flame spray pyrolysis device 10 ranges from 5 L/min to 70 L/min; more preferentially from 10 L/min to 50 L/min.

According to a particularly preferred embodiment of the invention, the correction factor q is between 1 and 2.2, more preferentially between 1.05 and 2, even more preferentially between 1.1 and 1.8, better still between 1.2 and 1.4; and the flow rate of inert gas (G2), more particularly nitrogen, injected into the flame spray pyrolysis device 10 ranges from 5 L/min to 70 L/min; more preferentially from 10 L/min to 50 L/min.

The preparation process according to the invention also comprises a step c. comprising the injection of a composition (B) comprising one or more precursors of element M′ until an upper coating layer 2 constituted of element M′ or of element M′ oxide(s) is obtained on the surface of said cerium oxide aggregates.

As indicated previously, according to the invention, the element M′ is chosen from selenium and the elements of columns 4, 13 and 14 of the Periodic Table of the Elements.

According to the invention, the element M′ is thus different from cerium.

Preferably, the element M′ is chosen from selenium, titanium, aluminium and the elements of column 14 of the Periodic Table of the Elements.

More preferentially, the element M′ is chosen from selenium, titanium, aluminium, carbon and silicon.

Most particularly preferably, the element M′ is chosen from carbon and silicon.

According to a preferred embodiment, the element M′ is silicon.

According to another embodiment of the invention, the element M′ is carbon.

Preferably, the precursor of element M′ comprises at least two M′ atoms and several M′-carbon covalent bonds. More preferentially, the precursor of element M′ comprises at least three M′ atoms and several M′-carbon covalent bonds.

More preferentially, the precursor of element M′ is chosen from hexa(di)(C₁-C₄)alkyldisiloxanes, such as hexadimethyldisiloxane, (di)(tri)(tetra)(C₁-C₄)alkoxysilanes, such as tetraethoxysilane, bis[(di)(tri)alkoxysilyl](C₁-C₄)alkanes, such as 1,2-bis(triethoxysilyl)ethane or 1,2-bis(trimethoxysilyl)ethane, (C₁-C₄)alkoxy(di)(tri)(C₁-C₄)alkylsilanes, such as methoxytrimethylsilane, hydrocarbon gases, such as acetylene, aluminium (di)(C₁-C₆)alkoxylates, aluminium (di)(C₁-C₆)alkylcarboxylates, such as aluminium diacetate hydroxide, (poly)(C₁-C₆)alkoxylate stannates, (poly)(C₁-C₆)alkylcarboxylate stannates, such as tetraacetate stannate, and mixtures thereof.

Even more preferentially, the precursor of element M′ is chosen from hexadimethyldisiloxane, tetraethoxysilane, 1,2-bis(triethoxysilyl)ethane, 1,2-bis(trimethoxysilyl)ethane, methoxytrimethylsilane and mixtures thereof.

According to a particular embodiment of the invention, composition (B) can be injected with an inert gas (G3) chosen, for example, from nitrogen, methane, argon, hydrogen, hydrogen sulfide and ammonia; preferably from nitrogen, methane, hydrogen and argon; and more preferentially from nitrogen and argon.

For example, nitrogen can be bubbled into composition (B), prior to its injection during step c. The flow rate of injection of composition (B) can subsequently be controlled by determination of the known pressure by a person skilled in the art, for instance the method defined by Scott, D. W.; Messerly, J. F.; Todd, S. S.; Guthrie, G. B.; Hossenlopp, I. A.; Moore, R. T.; Osborn, A. G.; Berg, W. T.; Mccullough, J. P., Hexamethyldisiloxane: chemical thermodynamic properties and internal rotation about the siloxane linkage, J. Phys. Chem., 1961, 65, 1320-6.

Preferably, the content of precursor(s) of element M′ in composition (B) injected during step c. of the process according to the invention is between 1% and 60% by volume, more preferentially between 5% and 30% by volume, relative to the total volume of composition (B).

Advantageously, composition (B) may also comprise one or more solvents. Preferably, the solvent(s) present in composition (B) are chosen from polar protic solvents other than water; and more preferentially from (C₁-C₈)alkanols. Even more preferentially, composition (B) comprises ethanol.

Preferably, the solvent(s) present in composition (B) are chosen from solvents that are combustible at the flame temperature of step c., preferably combustible at a temperature of between 200° C. and 600° C. and more preferentially between 300° C. and 400° C. Better still, the solvent(s) present in composition (B) have a boiling point greater than or equal to ambient temperature (25° C.) and more preferentially between 50° C. and 120° C.

During the process according to the invention, a molar atomic ratio (Ce/M′)_{injected} can be calculated. This ratio corresponds to the molar amount of cerium atoms injected during step b., on the one hand, to the molar amount of element M′ injected during step c., on the other hand.

Preferably, the molar atomic ratio (Ce/M′)_{injected} is greater than or equal to 0.25, more preferentially in the range from 0.25 to 120, even more preferentially from 0.25 to 99, better still in the range from 1 to 80; and even better still in the range from 2 to 20.

According to the invention, the flame spray pyrolysis device 10 is isolated from the external air, so that the amount of oxygen present in said device 10 is controlled, and more preferentially so that the oxygen present in said device 10 originates solely from said gas (G) and optionally from the mixture (P). In other words, atmospheric dioxygen cannot enter the combustion chamber(s) and react with composition (A) and the solvent(s).

Preferably, step b is performed in a first chamber 20 of the flame spray pyrolysis device 10 and step c is performed in a second chamber 30 of said device 10 in fluid communication with the first chamber 20.

As illustrated in FIGS. 2 and 3, said second chamber 30 is contiguous with the first chamber 20 and extends said first chamber. As a variant, provision might be made for the two chambers to be connected by a pipe.

The invention also relates to the coated cerium (sub)oxide particles of formula (I) or (I′) obtained according to the preparation process according to the invention described above.

### The Flame Spray Pyrolysis Device

The flame spray pyrolysis device that may be used to perform the preparation process of the invention preferably comprises a first chamber, a second chamber in fluid communication or connection to the first chamber, an injection system comprising a first feed, for example a first tube, opening into the first chamber and capable of delivering a first composition (A) and a first oxygen-containing gas (G) and a second feed, for example a second tube, opening into the first chamber and capable of delivering a mixture (P) comprising oxygen and one or more combustible gases, the first and second feeds being different from each other.

The device also comprises a third feed that is capable of delivering a second composition (B) comprising one or more precursors of element M′ into the second chamber.

The first and second chambers of said device are isolated from the external air, so that the amount of oxygen present in said device is controlled, and more preferentially so that the oxygen present in said first and second chambers originates solely from said first gas (G) and optionally from said mixture (P).

For example, in an entirely non-limiting manner, the second chamber is coaxial with the first chamber and, for example, arranged in the prolongation of said first chamber.

Advantageously, the first and second feeds are coaxial, the second feed at least partially surrounding the first feed.

According to one embodiment, the first chamber comprises two separate compartments, the first compartment comprising a first opening into which the injection system emerges and a second opening, on the side opposite the first opening, the second compartment at least partially surrounding the first compartment and being isolated from the external air, said second compartment being separated from the first compartment by a first partition.

For example, the first partition is porous so as to allow the passage of the gas into the first compartment. The second compartment is pressurized with a gas chosen, for example, from nitrogen, methane, argon, hydrogen, hydrogen sulfide and ammonia, or by heating.

For example, the device comprises an injector configured to inject a second gas into the second compartment of the first chamber and thus to pressurize said second compartment.

According to one embodiment, the second chamber comprises two separate compartments, the first compartment comprising a first opening, in fluid communication with or connected to the second opening of the first chamber, and a second opening, on the side opposite the first opening, the second compartment at least partially surrounding the first compartment and being isolated from the external air, said second compartment being separated from first compartment by a second partition and being equipped with a feed for feeding the second composition (B) into the second chamber.

For example, the device comprises an additional feed configured to inject a third gas into the second compartment of the second chamber and thus to pressurize said second compartment.

For example, the second partition is porous or perforated so as to allow the passage of the second composition (B) into the first compartment of the second chamber. The second compartment is pressurized with a third gas (G3) chosen, for example, from nitrogen, methane, argon, hydrogen, hydrogen sulfide and ammonia, or by heating.

For example, the device also comprises a collecting system, for example coaxial with the two chambers, arranged above the second chamber and configured to stop the particles while at the same time allowing the gases to pass. In other words, the collecting system is gas-permeable. For example, the collecting system comprises a filtration system fitted inside said collecting system and a pressure-reducing system configured to create a negative pressure inside the collecting system.

The injection system, the first chamber, the second chamber and the collecting system are assembled, for example by screwing or welding, so as to ensure perfect leaktightness of the device making it possible to prevent the access of external air into said device.

In an entirely non-limiting manner, the injection system, the first chamber, the second chamber and the collecting system are arranged in an enclosure so as to ensure perfect leaktightness of the device making it possible to prevent the access of external air into said enclosure. The interior of the enclosure is placed under negative pressure by means of the pressure-reducing system.

An example of a flame spray pyrolysis device 10 is illustrated in FIG. 2.

The flame spray pyrolysis device 10 comprises a first chamber 20 using composition (A) and the oxygen-containing gas (G), and a second chamber 30 using composition (B) comprising one or more precursors of element M′.

The flame spray pyrolysis device 10 also comprises an injection system 40 comprising a first tube 42 emerging in the first chamber 20 and delivering composition (A) and the oxygen-containing gas (G), and a second tube 44 emerging in the first chamber 20 and delivering the “premix” mixture (P) comprising oxygen and one or more combustible gases such as methane. The second tube 44 provides a flame necessary for the ignition of the compounds coming from the first tube 42.

The first and second tubes 42, 44 are separate from each other.

As illustrated, the first and second tubes 42, 44 are coaxial and the second tube 44 at least partially surrounds the first tube 42.

In an entirely non-limiting manner, the injection system 40 of the device 10 also comprises an additional feed 46, into the first chamber 20, of an inert gas, for instance nitrogen. The additional feed 46 may be in the form of a porous part, from which the inert gas can emerge at a pressure of between 2 and 20 bar (i.e. between 2×10⁵ and 20×10⁵ Pa).

Composition (A), the oxygen-containing gas (G) and the combustible (P) coming from the injection system 40 are incinerated in the first chamber 20.

As illustrated, the first chamber 20 comprises two separate compartments 22, 24. The first compartment 22 comprise a first lower opening 22a into which the injection system 40 emerges and a second upper opening 22b, on the side opposite the first opening 22a.

The second compartment 24 surrounds the first compartment 22 and is isolated from the external air. The second compartment 24 is separated from the first compartment 22 by a gas-permeable partition 26.

The second compartment 24 comprises an upper wall, a lower wall and side walls (not referenced) forming a closed housing isolated from the external air.

The second compartment 24 is pressurized with a gas (G2) chosen, for example, from nitrogen, methane, argon, hydrogen, hydrogen sulfide and ammonia. The gas (G2) is injected into the second compartment 24 via an injector 28. For example, the injector 28 comprises a single tube emerging into the second compartment 24. As a variant, the injector 28 may comprise two or more tubes emerging into the second compartment 24. The tubes may or may not be evenly spaced over the circumference of the second compartment 24.

The partition 26 for separation of the two compartments 22, 24 is configured so as to allow the passage of the gas (G2) into the first compartment 22. For example, the partition 26 is made of porous material. The porosity of the partition 26 is, for example, of between 10 μm and 100 μm.

The first chamber 20 has a height H1, for example of between 10 cm and 1 m.

The second chamber 30 is configured so as to use composition (B) comprising one or more precursors of element M′.

As illustrated, the second chamber 30 comprises two separate compartments 32, 34. The first compartment 32 comprises a first lower opening 32a coinciding with the second opening 22b of the first chamber 20, and a second upper opening 32b, on the side opposite the first opening 32a.

As a variant, the first lower opening 32a might be offset laterally from the second opening 22b of the first chamber 20. Provision might also be made for the first chamber 20 to be connected to the second chamber 30 by a pipe.

The second compartment 34 surrounds the first compartment 32 and is isolated from the external air. The second compartment 34 is separated from the first compartment 32 by a gas-permeable partition 36.

The second compartment 34 comprises an upper wall, a lower wall and side walls (not referenced) forming a closed housing isolated from the external air.

The second compartment 34 is equipped with a feed 38 for feeding composition (B) into the second chamber 30.

The feed 38 is pressurized with a gas (G3) chosen, for example, from nitrogen, methane, argon or hydrogen, or by heating composition (B). For example, the feed 38 comprises a single tube emerging into the second compartment 34. As a variant, the feed 38 comprises two or more tubes emerging into the second compartment 34. The tubes may or may not be evenly spaced over the circumference of the second compartment 34.

The partition 36 for separation of the two compartments 32, 34 is configured so as to allow the passage of composition (B) from the second compartment 34 to the first compartment 32. For example, the partition 36 comprises a plurality of perforations (not shown), from approximately 0.1 mm to 0.5 mm, in number from 1 to 10 perforations per cm² of the separating partition 36.

The second chamber 30 has a height H2, for example of between 10 cm and 1 m.

Preferably, the height H1 of the first chamber 20 is equal to the height H2 of the second chamber 30 plus or minus 10%. Preferably, the dimensions of the first chamber 20 are equal to the dimensions of the second chamber 30.

The flame spray pyrolysis device 10 also comprises a collecting system 50 configured to stop the particles while at the same time allowing the gases to pass.

The collecting system 50 is in this instance coaxial with the two chambers 20, 30 and arranged above the second chamber 30. As a variant, provision might be made for the collecting system 50 to be offset laterally from the chambers 20, 30.

The collecting system 50 is delimited radially by one or more side partitions 52 and axially by a lower wall 54 comprising an opening 54a emerging into the second chamber 30 and an upper wall 55 on the side opposite the lower wall 54.

The collecting system 50 also comprises a filtration system 56 fitted inside said collecting system between the side walls 52, and a pressure-reducing system 58, for instance a pump, mounted on the upper wall 55 of said system 50.

The pump 58 is configured to create a negative pressure inside the collecting system 50 so as to isolate the chambers 20, 30 from the external air. Advantageously, the negative pressure inside the collecting system 50 is of the order of 0.5 to 0.8 bar (i.e. between 5×10⁴ and 8×10⁴ Pa).

In an entirely non-limiting manner, the collecting system 50 is spaced out axially from the second chamber 30 by a spacer 60.

As illustrated in FIG. 2, the injection system 40, the first chamber 20, the second chamber 30 and the collecting system 50, or even the spacer 60 when it is present, are assembled, for example by screwing or welding, so as to ensure perfect leaktightness of the device 10, and notably of the chambers 20, 30, making it possible to prevent the access of external air into said device 10.

The embodiment illustrated in FIG. 3, in which the same elements bear the same references, differs from the embodiment illustrated in FIG. 2 only in that the injection system 40, the first chamber 20, the second chamber 30 and the collecting system 50, or even the spacer 60 when it is present, are arranged in an enclosure 70, so as to ensure perfect leaktightness of the device 10, and notably of the chambers 20, 30, making it possible to prevent the access of external air into said enclosure 70. The interior of the enclosure 70 is placed under negative pressure by means of the pump 58.

According to a preferred embodiment of the device, said device has an axis of symmetry A which passes through the centre/middle of the injection system 40 and through the centre/middle of the collecting system 50. More preferentially, the device is symmetrical and notably cylindrical, passing through said axis of symmetry A.

### The Composition

Another subject of the invention relates to a composition, preferably a cosmetic composition, comprising one or more coated cerium suboxide particles of formula (I′) as described above, and/or preferably obtained via the preparation process according to the invention.

The composition according to the invention is intended to be applied to keratin materials, preferably the skin (notably the face) and/or the hair, in order to dye and/or make up the keratin materials. An optional step of drying the keratin materials may be performed.

The composition according to the invention may be in various presentation forms. Thus, the composition according to the invention may be in the form of a powder (pulverulent) composition or a liquid composition, or in the form of a milk, a cream, a paste or an aerosol composition.

The compositions according to the invention are in particular cosmetic compositions, i.e. the material(s) of the invention are in a cosmetically acceptable carrier. The term “cosmetically acceptable carrier” means a medium that is suitable for application to keratin materials, notably human keratin materials such as the skin, said cosmetic carrier generally consisting of water or of a mixture of water and of one or more organic solvents or of a mixture of organic solvents.

The composition according to the invention is advantageously an aqueous composition.

Preferably, the composition comprises water in a content notably between 5% and 95% inclusive, relative to the total weight of the composition.

For the purposes of the invention, the term “organic solvent” means an organic substance that is capable of dissolving another substance without chemically modifying it.

Examples of organic solvents that may be mentioned include lower C₂-C₆ alkanols, such as ethanol and isopropanol; polyols and polyol ethers, for instance 2-butoxyethanol, propylene glycol, propylene glycol monomethyl ether and diethylene glycol monoethyl ether and monomethyl ether, and also aromatic alcohols, for instance benzyl alcohol or phenoxyethanol, and mixtures thereof.

Preferably, the organic solvents are present in the composition according to the invention in a content inclusively between 0.1% and 40% by weight approximately, relative to the total weight of the composition, more preferentially between 1% and 30% by weight approximately and even more particularly inclusively between 5% and 25% by weight, relative to the total weight of the composition.

The compositions of the invention may contain a fatty phase and may be in the form of direct or inverse emulsions.

The composition according to the invention may be prepared according to the techniques well known to those skilled in the art, in the form of a simple or complex emulsion (oil-in-water, abbreviated to O/W, water-in-oil or W/O, oil-in-water-in-oil or O/W/O, or water-in-oil-in-water or W/O/W), such as a cream, a milk or a cream gel.

According to a particular embodiment of the invention, the composition according to the invention may also be in the form of an anhydrous composition, for instance in the form of an oil. The term “anhydrous composition” means a composition containing less than 2% by weight of water, preferably less than 1% by weight of water and even more preferentially less than 0.5% by weight of water, relative to the total weight of the composition, and indeed even is free of water. In compositions of this type, the water that may be present is not added during the preparation of the composition but corresponds to the residual water contributed by the mixed ingredients.

The particle(s) according to the invention may also be in dry form (powder, flakes, plates), as a dispersion or as a liquid suspension or as an aerosol. The particle(s) according to the invention may be used as is or mixed with other ingredients.

Preferably, the compositions of the invention contain between 0.1% and 40% by weight of particles according to the invention, more preferentially between 0.5% and 20% by weight, even more preferentially between 1% and 10% by weight and better still between 1.5% and 5% by weight, relative to the total weight of the composition.

The compositions of the invention may be used in single application or in multiple application. When the compositions of the invention are intended for multiple application, the content of particles of the invention is generally lower than in compositions intended for single application.

For the purposes of the present invention, the term “single application” means a single application of the composition, this application possibly being repeated several times per day, each application being separated from the next one by one or more hours, or an application once a day, depending on the need.

For the purposes of the present invention, the term “multiple application” means application of the composition repeated several times, in general from 2 to 5 times, each application being separated from the next one by a few seconds to a few minutes. Each multiple application may be repeated several times per day, separated from the next one by one or more hours, or each day, depending on the need.

Another subject of the invention is a process for treating keratin materials, notably human keratin materials such as the skin, by application to said materials of a composition as defined previously, preferably by 1 to 5 successive applications, leaving to dry between the layers, the application(s) being sprayed or otherwise.

According to one embodiment of the invention, the multiple application is performed on the keratin materials with a drying step between the successive applications of the cosmetic compositions comprising the metal oxide particle(s) of the invention. The drying step between the successive applications of the cosmetic compositions comprising at least one metal oxide particle of the invention may be performed in the open air or artificially, for example with a hot-air drying system such as a hairdryer.

Another subject of the invention is the composition according to the invention, preferably a cosmetic composition, for its use in protecting the skin, preferably human skin, against visible radiation (i.e. of wavelengths between 400 nm and 800 nm) and/or ultraviolet radiation (i.e. of wavelengths between 100 nm and 400 nm), UV-A radiation (i.e. of wavelengths between 320 nm and 400 nm) and/or UV-B radiation (i.e. of wavelengths between 280 nm and 320 nm), preferably UV-A radiation. The compositions according to the invention make it possible to screen out solar radiation effectively, with a broad spectrum notably for UV-A radiation (including long UV-A), while at the same time being particularly stable over time under UV exposure.

The composition according to the present invention may optionally comprise one or more additional UV-screening agents, other than the metal oxide particle according to the invention, chosen from hydrophilic, lipophilic or insoluble organic UV-screening agents and/or one or more mineral pigments. It will preferentially consist of at least one hydrophilic, lipophilic or insoluble organic UV-screening agent.

A subject of the invention is also the use of the cerium oxide particles as described previously and/or obtained via the preparation process as described previously, for the formulation of cosmetic or pharmaceutical compositions, in particular with an antiperspirant action or for regulating the pH of the skin, or intended to protect the skin against visible and/or ultraviolet radiation or to modify the appearance of the skin.

Another subject of the invention is the use of one or more cerium oxide particles as defined previously, as screening agent for UV-A and/or UV-B, preferably UV-A, for protecting keratin materials, notably the skin.

The examples that follow serve to illustrate the invention without, however, being limiting in nature.

### EXAMPLES

### Example 1

1.1 In a first stage, a composition (A) was prepared from cerium (III) ethylhexanoate (500 mM) in xylene.

Uncoated cerium oxide particles P1 (higher oxidation number) were then prepared via a conventional FSP Prep 1 preparation process with the previously prepared composition (A) (outside the invention).

The parameters of the Prep 1 process are as follows:
ratio (composition (A)/O₂)=5 mL/min of composition (A) and 5 L/min of gas (O₂),
a gas mixture to ensure the flame of 1 L/min of methane and 2 L/min of dioxygen
a stream of inert gas (G2) of 10 L/min of nitrogen is injected into the FSP device.
A φ=0.7 is used to adjust the oxygen flow rate.
1.2 Uncoated cerium suboxide particles P2 were then prepared via an FSP Prep 2 preparation process with the previously prepared composition (A)(outside the invention).

The parameters of the Prep 2 process are as follows:
ratio (composition (A)/O₂)=5 mL/min of composition (A) and 5 L/min of gas (O₂),
a gas mixture to ensure the flame of 1 L/min of methane and 2 L/min of dioxygen
a stream of inert gas (G2) of 20 L/min of nitrogen is injected into the FSP device.
A φ=1.2 is used to adjust the oxygen flow rate.
1.3 Cerium suboxide particles coated with silicon dioxide P3 were then prepared in a double-chamber FSP device via the Prep 3 preparation process according to the invention with a composition (A) comprising cerium (III) ethylhexanoate (400 mM) in xylene and a composition (B) comprising hexadimethyldisiloxane (100 mM in xylene) and ethanol in a weight ratio of 3:1 (invention).

The parameters of the Prep 3 process are as follows:
composition (A) and the oxygen-containing gas (G) are injected into the first chamber of the FSP device, according to a (composition (A)/O₂) ratio=5 mL/min of composition (A) and 5 L/min of gas (O₂),
a gas mixture to ensure the flame of 1 L/min of methane and 2 L/min of dioxygen,
a stream of inert gas (G2) of 25 L/min of nitrogen is injected into the first chamber of the double-chamber FSP device,
composition (B) is injected into the second chamber of the FSP device by means of a 3 L/min nitrogen stream.
A q=1.3 is used to adjust the oxygen flow rate.

Once the P3 particles were prepared, it was observed that the cerium suboxide particles P3 obtained were crystalline.

Moreover, the P3 particles obtained according to the Prep 3 process according to the invention are coated with a top layer of silicon dioxide about 5 nm thick, and have an atomic ratio (Ce/Si) particle of 2.

The BET specific surface area of the particles P3 is 62 m²/g.

The particles P3 have a number-average diameter equal to 15 nm.

The oxidation of particles P1 to P3 was monitored by X-ray diffraction.
1.4 The optical properties of particles P1 to P3 were then evaluated.

It was observed that the particles P3 according to the invention have excellent power for screening out UV-A radiation.

It was also observed that the particles P3 according to the invention have a UV-screening power 2.47 times greater (for an equal volume) than the screening power of the uncoated cerium oxide particles P1 (outside the invention).

In addition, it was observed that the particles P3 according to the invention have a UV-screening power 1.62 times greater (for an equal volume) than the screening power of the uncoated cerium suboxide particles P2 (outside the invention).

### Example 2

2.1 Carbon-coated cerium suboxide particles P4 were then prepared in a single-chamber FSP device via the Prep 4 preparation process according to the invention with a composition (A) comprising cerium (III) ethylhexanoate (500 mM) in xylene and a composition (B) consisting of acetylene (invention).

The parameters of the Prep 4 process are as follows:
composition (A) and the oxygen-containing gas (G) are injected into the FSP device at a ratio (composition (A)/O₂)=5 mL/min of composition (A) and 5 L/min of gas (O₂)
a gas mixture to ensure the flame of 1 L/min of methane and 2 L/min of dioxygen
a stream of inert gas (G2) of 50 L/min of nitrogen
acetylene (composition (B)) is injected into the FSP device at 2.5 L/min.
A φ=1.9 is used to adjust the oxygen flow rate.
2.2 Carbon-coated cerium suboxide particles P5 were prepared in a double-chamber FSP device via the Prep 5 preparation process according to the invention with a composition (A) comprising cerium (III) ethylhexanoate (500 mM) in xylene and a composition (B) consisting of acetylene (invention).

The parameters of the Prep 5 process are as follows:
composition (A) and the oxygen-containing gas (G) are injected into the first chamber of the FSP device, according to a (composition (A)/O₂) ratio=5 mL/min of composition (A) and 5 L/min of gas (O₂),
a gas mixture to ensure the flame of 1 L/min of methane and 2 L/min of dioxygen is injected into the first chamber of the FSP device
a stream of inert gas (G2) of 25 L/min of nitrogen is injected into the first chamber of the FSP device
acetylene (composition (B)) is injected into the second chamber of the FSP device at 2.5 L/min.
A φ=1.6 is used to adjust the oxygen flow rate.

Once the particles P4 and P5 were prepared, it was observed that the cerium suboxide particles P4 and P5 obtained were crystalline.

Moreover, the particles P4 obtained according to the Prep 4 process according to the invention are coated with a top layer of carbon about 1.5 nm thick, and have an atomic ratio (Ce/C)_{particle} of 0.3.

The BET specific surface area of the particles P4 is 74 m²/g.

The particles P4 have a number-average diameter equal to 14 nm.

The particles P5 obtained via the Prep 5 process according to the invention are coated with a top layer of carbon about 1 nm thick, and have an atomic ratio (Ce/C)_{particle} of 0.57.

The BET specific surface area of the particles P5 is 70 m²/g.

The particles P5 have a number-average diameter equal to 16 nm.

The oxidation of the particles P4 and P5 was monitored by X-ray diffraction.
2.3 The optical properties of the particles P4 and P5 were then evaluated.

It was observed that the particles P4 and P5 according to the invention have excellent power for screening out UV-A radiation.

It was also observed that the particles P4 according to the invention have a UV-screening power 2.04 times greater (for an equal volume) than the screening power of the uncoated cerium oxide particles P1 (outside the invention).

In addition, it was observed that the particles P5 according to the invention have a UV-screening power 2.35 times greater (for an equal volume) than the screening power of the uncoated cerium suboxide particles P1 (outside the invention).

1. Particle comprising:
(i) a core (1) constituted of at least one cerium suboxide of formula (I′):
CeO₂₋ₓ (I′)
in which x represents a non-integer number strictly between 0 and 2; and
(ii) an upper coating layer (2), covering the surface of said core (1), constituted of at least one compound of formula (II):
M′ₚO_{q} (II)
in which:
M′ is an element chosen from selenium and the elements in columns 4, 13 and 14 of the Periodic Table of the Elements;
p represents an integer greater than or equal to 1,
q represents an integer greater than or equal to 0.

2. Particle according to claim 1, characterized in that:
p represents an integer ranging from 1 to 4, preferably equal to 1 or 2, more preferentially equal to 1; and/or
q represents an integer ranging from 0 to 4.

3. Particle according to claim 1, characterized in that the element M′ is chosen from selenium, titanium, aluminium and the elements of column 14 of the Periodic Table of the Elements; preferably from selenium, titanium, aluminium, carbon and silicon; and more preferentially from carbon and silicon.

4. Particle according claim 1, characterized in that the compound(s) of formula (II) are chosen from carbon, SiO₂, SnO₂ and Al₂O₃; preferably from carbon and SiO₂.

5. Particle according to claim 1, characterized in that the molar atomic ratio (Ce/M′) of the particle is strictly greater than 0.2; preferably greater than or equal to 1; more preferentially in the range from 1 to 10.

6. Particle according to claim 1, characterized in that the number-average thickness dₘ of the upper coating layer (2), measured by transmission electron microscopy, is in the range from 1 to 20 nm, preferably from 1 to 10 nm and more preferentially from 2 to 6 nm.

7. Particle according to claim 1, characterized in that the number-average diameter of the particles, as determined by transmission electron microscopy, is in the range from 4 to 5000 nm, preferably from 10 to 3000 nm and more preferentially from 30 to 1000 nm.

8. Particle according to claim 1, characterized in that the upper coating layer (2) of the particle covers at least 90% of the surface of the core (1); preferably covers the entire surface of the core (1).

9. Process for preparing one or more coated cerium oxide or suboxide particles comprising:
(i) a core (1) constituted of at least one cerium oxide of formula (I):
CeO₂₋ₓ (I)
in which x is equal to 0 or represents a non-integer number strictly between 0 and 2; and
(ii) an upper coating layer (2), covering the surface of said core (1), constituted of at least one compound of formula (II):
M′ₚO_{q} (II)
in which:
M′ is an element chosen from selenium and the elements in columns 4, 13 and 14 of the Periodic Table of the Elements;
p represents an integer greater than or equal to 1,
q represents an integer greater than or equal to 0;
comprising at least the following steps:
a. preparing a composition (A) by adding one or more cerium precursors in a combustible solvent or in a mixture of combustible solvents; and then
b. in a flame spray pyrolysis device (10), forming a flame by injecting composition (A) and an oxygen-containing gas (G) until cerium oxide aggregates of formula (I) are obtained; and then
c. injecting a composition (B) comprising one or more precursors of element M′ until an upper coating layer (2) constituted of element M′ or of element M′ oxide of formula (II) is obtained on the surface of said cerium oxide aggregates; and
said flame spray pyrolysis device (10) being isolated from the exterior air, so that the amount of oxygen present in said device is controlled.

10. Process according claim 9, characterized in that composition (A) comprises a mixture of combustible solvents, preferably comprising at least two of the following combustible solvents: 2-ethylhexanoic acid, toluene, absolute ethanol and diethylene glycol monobutyl ether; more preferentially the combustible solvent mixture consists of at least 5% by volume of 2-ethylhexanoic acid, at least 5% by volume of toluene, at least 5% by volume of absolute ethanol and at least 5% by volume of diethylene glycol monobutyl ether, relative to the total volume of the combustible solvent mixture.

11. Process according to claim 9, characterized in that the element M′ is chosen from selenium, titanium, aluminium and the elements of column 14 of the Periodic Table of the Elements; preferably from selenium, titanium, aluminium, carbon and silicon; and more preferentially from carbon and silicon.

12. Process according to claim 9, characterized in that, taken together or separately:
the molar atomic ratio (Ce/M′)_{injected} is greater than or equal to 0.25, preferably in the range from 0.25 to 120, more preferentially from 0.25 to 99, even more preferentially from 1 to 80; and better still from 2 to 20; and/or
step b is performed in a first chamber (20) of a flame spray pyrolysis device (10) and step c is performed in a second chamber (30) of the device (10) in fluid communication with the first chamber (20); and/or
composition (B) comprises one or more solvents; preferably, the solvent(s) are chosen from polar protic solvents other than water; more preferentially from (C₁-C₈)alkanols; and better still the solvent is ethanol; and/or
the molar amount of oxygen-containing gas (G) injected during step b is strictly less than the molar amount of oxygen-containing gas required to cause composition (A) to react with the oxygen in a stoichiometric ratio.

13. Process according to claim 9, characterized in that the flame projection pyrolysis device (10) is pressurized by means of an inert gas (G2), preferably chosen from nitrogen, methane, argon, hydrogen, hydrogen sulfide and ammonia; more preferentially from nitrogen, methane, hydrogen and argon; even more preferentially from nitrogen and argon, and better still nitrogen.

14. Particle according to claim 1, characterized in that it is obtained via the process as defined in any one of claims 9 to 13.

15. Composition, preferably cosmetic, comprising one or more particles as defined in claim 1.

16. Composition according to claim 15, for its use in protecting the skin, preferably human skin, against visible and/or ultraviolet radiation, UV-A and/or UV-B.
